(19) United States
(12) Patent Application Publication
(10) Pub. No.: US 2025/0185899 A1
(43) Pub. Date: Jun. 12, 2025

(54) ENDOSCOPE, BENDING TUBE, AND METHOD FOR MANUFACTURING BENDING TUBE

(71) Applicant: OLYMPUS MEDICAL SYSTEMS CORP., Tokyo (JP)

(72) Inventors: Kohki KAMATA, Tokyo (JP); Hiroyuki SEKI, Tokyo (JP); Kentaro YAMADA, Tokyo (JP); Norimitsu NAGAYAMA, Tokyo (JP)

(73) Assignee: OLYMPUS MEDICAL SYSTEMS CORP., Tokyo (JP)

(21) Appl. No.: 18/968,589

(22) Filed: Dec. 4, 2024

Related U.S. Application Data
(60) Provisional application No. 63/607,233, filed on Dec. 7, 2023.

(51) Int. Cl. A61B 1/005; A61B 1/00

(52) U.S. Cl. CPC A61B 1/0055; A61B 1/0011; A61B 1/0057

(57) ABSTRACT

An endoscope includes: a bending tube, the bending tube including: a plurality of bending pieces; a plurality of hinges linking two adjacent bending pieces of the plurality of bending pieces in a freely pivotable manner in a long axis direction; and at least one elastic member including a plurality of fixing regions, fixed to a circumferential region of the plurality of bending pieces, respectively, wherein a first length of the at least one elastic member between a pair of fixing regions in the long axis direction is greater than a second length between a respective pair of circumferential regions in the long axis direction.

### RELATED APPLICATION DATA

This application is based on and claims priority under 35 U.S.C. § 119 to U.S. Provisional Application No. 63/607,233, filed Dec. 7, 2023, the entire contents of which are incorporated herein by reference.

### BACKGROUND

### 1. Field

The present disclosure relates to an endoscope including an insertion portion in which a bending tube is disposed, the bending tube disposed in the insertion portion of the endoscope, and a method for manufacturing the bending tube disposed in the insertion portion of the endoscope.

### 2. Description of the Related Art

A bending tube of an endoscope is manufactured by linking a plurality of bending pieces each by hinges in the long axis direction. There are gaps between the plurality of bending pieces, and each of the plurality of bending pieces turns freely without being affected by the movement of the other plurality of bending pieces. Outer circumferential surfaces of the plurality of bending pieces are covered with an outer tube made of, e.g., rubber.

Japanese Patent Publication No. 2008-099827 discloses a structure in which an outer tube is deformed outward between a plurality of bending pieces in order to prevent the outer tube from being sandwiched between the plurality of bending pieces.

Japanese Patent Publication No. 2010-000311 discloses a bending tube in which an inclined surface is provided on an outer circumferential surface of a plurality of bending piece in order to prevent an outer tube from being sandwiched between the bending pieces.

### SUMMARY

An endoscope of an embodiment of the present disclosure includes a bending tube, the bending tube including: a plurality of bending pieces; a plurality of hinges linking two adjacent bending pieces of the plurality of bending pieces in a freely pivotable manner in a long axis direction; and at least one elastic member including a plurality of fixing regions, fixed to a circumferential region of the plurality of bending pieces, respectively, wherein a first length of the at least one elastic member between a pair of fixing regions in the long axis direction is greater than a second length between a respective pair of circumferential regions in the long axis direction.

A bending tube of an embodiment of the present disclosure includes: a plurality of bending pieces; a plurality of hinges that link two adjacent bending pieces of the plurality of bending pieces in a freely pivotable manner in a long axis direction; and at least one elastic member including a plurality of fixing regions, fixed to a circumferential region of the plurality of bending pieces, respectively, wherein a first length of the at least one elastic member between a pair of fixing regions in a long axis direction is greater than a second length between a respective pair of circumferential regions in the long axis direction.

A method for manufacturing a bending tube of an embodiment of the present disclosure includes: linking a plurality of bending pieces to each other in a freely pivotable manner in a long axis direction using a plurality of hinges, to produce a bending tube main body; disposing at least one elastic member over the bending tube main body; deforming the at least one elastic member by application of a compressive force in the long axis direction; and while the at least one elastic member is deformed, fixing the at least one elastic member at two or more fixing regions to circumferential surfaces of respective bending pieces of the plurality of bending pieces.

### BRIEF DESCRIPTION OF THE DRAWINGS

FIG. 1 is a perspective view of an endoscope of a first embodiment.

FIG. 2 is a perspective view of a bending tube main body of a bending tube of the first embodiment.

FIG. 3 is a perspective view of a bending piece of the bending tube of the first embodiment.

FIG. 4 is a perspective view of the bending tube of the first embodiment.

FIG. 5 is a cross-sectional view of the bending tube of the first embodiment.

FIG. 6A is a partial cross-sectional view of an elastic member of the bending tube of the first embodiment.

FIG. 6B is a partial cross-sectional view of the elastic member of the bending tube of the first embodiment.

FIG. 6C is a partial cross-sectional view of the elastic member of the bending tube of the first embodiment.

FIG. 7 is a flow chart of a method for manufacturing the bending tube of the first embodiment.

FIG. 8 is a partial cross-sectional view of an elastic member of a bending tube of Modification 1 of the first embodiment.

FIG. 9 is a partial cross-sectional view of an elastic member of a bending tube of Modification 2 of the first embodiment.

FIG. 10 is a partial cross-sectional view of an elastic member of a bending tube of Modification 3 of the first embodiment.

FIG. 11A is a cross-sectional view of an elastic member of a bending tube of Modification 4 of the first embodiment.

FIG. 11B is a cross-sectional view of the elastic member of the bending tube of the Modification 4 of the first embodiment.

FIG. 12 is a partial cross-sectional view of an elastic member of a bending tube of Modification 5 of the first embodiment.

FIG. 13 is an exploded perspective view of a bending tube of a second embodiment.

FIG. 14 is a cross-sectional view of a bending tube of a third embodiment.

FIG. 15 is a cross-sectional view of a bearing of the bending tube of the third embodiment.

FIG. 16 is a cross-sectional view of a bending tube of a fourth embodiment.

FIG. 17 is a perspective view of a measuring apparatus for a bending tube of a reference example.

FIG. 18 is a cross-sectional view for describing a method for measuring the bending tube of the reference example.

FIG. 19 is a perspective view for describing the method for measuring the bending tube of the reference example.

FIG. 20 is an example of a measurement result of the bending tube of the reference example.

### DETAILED DESCRIPTION

### First Embodiment

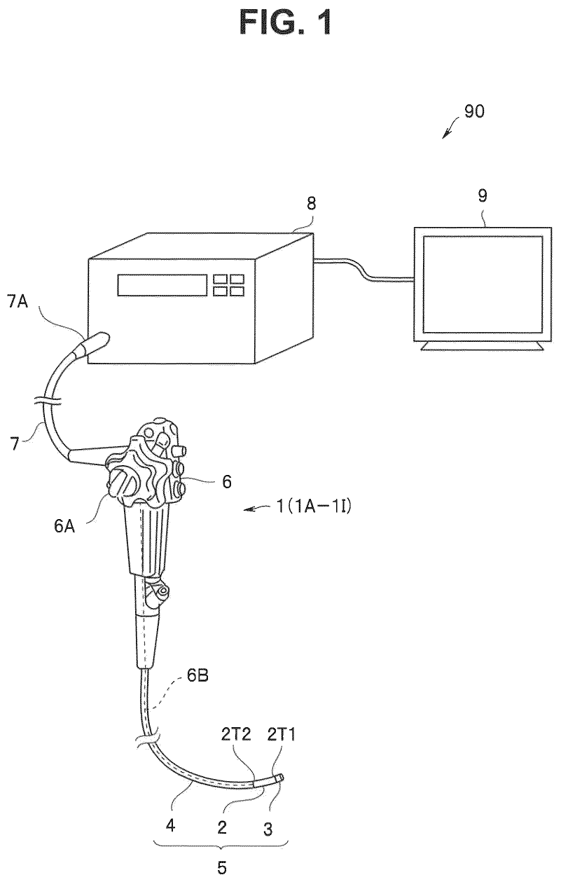

As shown in FIG. 1, an endoscope 1 of this embodiment configures an endoscope system 90 together with a processor 8 and a monitor 9.

In the following description, the drawings based on each embodiment are schematic. The relationship between the thickness and width of each part, as well as the ratio of the thickness, the relative angle, etc., of each part are different from the actual ones. The drawings also contain parts in which dimensional relationships and ratios are different from each other. Illustration of some components are omitted.

The endoscope 1 includes an insertion portion 5, a grasping portion 6 disposed on a proximal end portion side of the insertion portion 5, a universal cord 7 extended from the grasping portion 6, and a connector 7A disposed on the proximal end portion side of the universal cord 7. The insertion portion 5 includes a distal end portion 3, a bending tube 2 that is freely bendable and for changing the direction of the distal end portion 3, and a flexible tube 4 that is a proximal end tube. An image pickup unit (not shown) is disposed in the distal end portion 3. The bending tube 2 includes a first end 2T1 and a second end 2T2. The bending tube 2 has the first end 2T1 fixed to the distal end portion 3 and the second end 2T2 fixed to the flexible tube 4. The grasping portion 6 is disposed with a rotating angle knob 6A that is connected to an operation wire 6B that is inserted through the flexible tube 4 and that is an operation portion for the surgeon to operate the bending tube 2.

The universal cord 7 is connected to the processor 8 by the connector 7A. The processor 8 controls the entire endoscope system 90, and processes an image pickup signal output by the image pickup unit and outputs a resulting signal as an image signal. The monitor 9 displays the image signal output by the processor 8 as an endoscopic image.

The endoscope 1 is a flexible endoscope, but the endoscope of the embodiment may be a rigid endoscope whose proximal end tube is a rigid straight tube. A single endoscope having the processor 8 and the monitor 9 at the grasping portion 6 may configure the endoscope system. The endoscope 1 may be used for medical or industrial purposes.

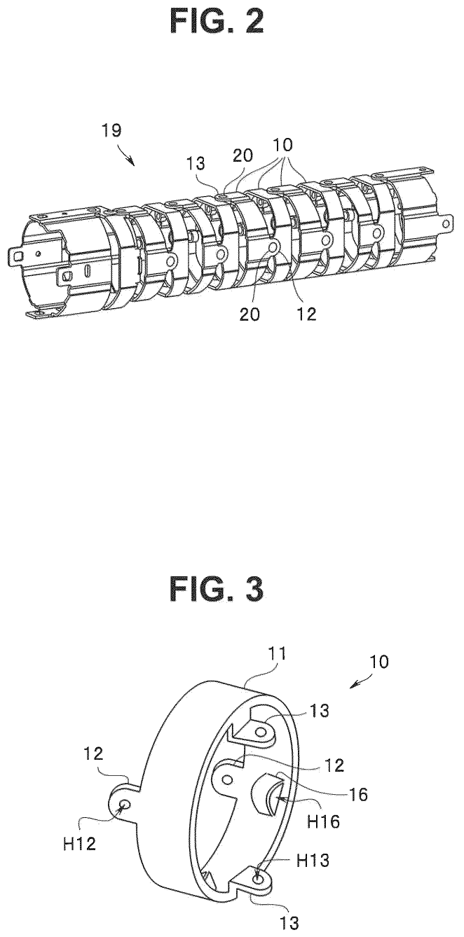

As shown in FIG. 2, the bending tube 2 includes a bending tube main body 19 having a substantially cylindrical shape and consisting of a plurality of bending pieces 10 linked in a longitudinal axis direction of the insertion portion 5. Although the present embodiments are described and illustrated with regard to separately formed bending pieces pivotably connected relative to each other at hinges, the present invention is also applicable to bending tubes having a series of slits where the bending pieces and hinges are integrally formed. The series of slits forming the bending pieces between such slits and forming hinges at such slits such that the bending pieces pivot relative to each other.

As shown in FIG. 3, the bending piece 10 includes a bending piece main body 11 having a cylindrical shape. At a distal end portion of the bending piece main body 11, two protruding pieces 12, each being a part of an outer circumferential surface of the bending piece main body 11 protruding toward the distal end, are placed at positions 180 degrees apart in the circumferential direction. At a proximal end portion of the bending piece main body 11, two protruding pieces 13, each being a part of the outer circumferential surface of the bending piece main body 11 protruding toward the proximal end, and each being formed by providing a step having approximately a plate thickness of the protruding piece 12, are placed at positions 180 degrees apart in the circumferential direction. Here, the two protruding pieces 12 on the distal end side and the two protruding pieces 13 on the proximal end side are placed at positions shifted by 90 degrees in the circumferential direction.

The plurality of bending pieces 10 arranged in a row in the bending tube main body 19 are linked in a pivotable manner. The two protruding pieces 13 on the proximal end side of the bending piece 10 on the distal end side and the two protruding pieces 12 on the distal end side of the bending piece 10 on the proximal end side are linked by respective hinges 20 inserted into respective holes H12 and H13 of the protruding pieces 12 and 13. As a result, the bending piece 10 on the distal end side and the bending piece 10 on the proximal end side are pivotally supported in a pivotable manner around the hinges 20, forming a support shaft portion with the hinge 20 serving as a turning support shaft.

In other words, two adjacent bending pieces 10 of the plurality of bending pieces 10 are linked in a freely pivotable manner in the long axis direction by four hinges 20.

The two protruding pieces 12 on the distal end side of the bending piece 10 at the first end 2T1 of the bending tube main body 19 are similarly and respectively linked by the hinges 20 to two protruding pieces each protruding rearward at the proximal end portion of the distal end portion 3, and are pivotally supported in a pivotable manner around the hinges 20. In addition, the two protruding pieces 13 on the proximal end side of the bending piece 10 at the second end 2T2 of the bending tube main body 19 are similarly and respectively linked by the hinges 20 to two protruding pieces each protruding forward at the distal end portion of the flexible tube 4, and are pivotally supported in a pivotable manner around the hinges 20.

Four wire guides 16 are provided on an inner circumferential surface of the bending piece 10. The operation wire 6B (see FIG. 1) is inserted through each of holes H16 of the four wire guides 16. The holes H16 each have an inner diameter greater than an outer diameter of the operation wire 6B. A distal end of the operation wire 6B is fixed to the bending piece 10 at the first end 2T1. By a turn operation of the angle knob 6A of the grasping portion 6, the operation wire 6B is pulled and the orientation of the bending tube 2 changes in four directions (up/down/left/right).

In manufacturing the bending tube 2, the plurality of bending pieces 10 provided consecutively are not restricted in the respective positions, thus freely turning in directions other than those intended by a bending operation. This caused the total length of the manufactured bending tube 2 to be outside the specified range, causing a risk of reducing the accuracy of angle adjustment, resulting in difficulty to manufacture.

The angle knob 6A maintains a state of being turn operated. Straightening the bending tube 2 that is bent necessitates the turn operation in an opposite direction, thus complicating the operation of the endoscope.

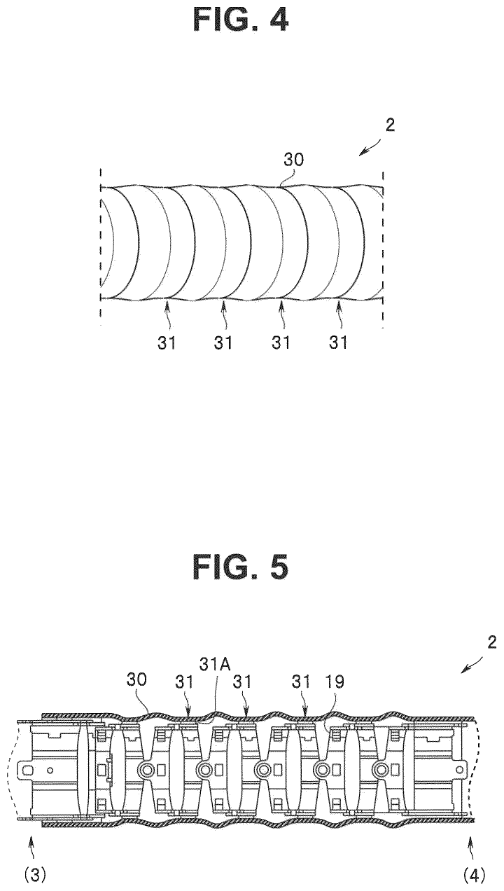

As shown in FIGS. 4 and 5, the bending tube 2 includes an outer tube 30 that covers the outer circumferential surfaces of the plurality of bending pieces 10 linked (bending tube main body 19). The outer tube 30 is an elastic member made of a cylindrical rubber.

Covering the outer circumferential surface of the bending tube main body 19 with an outer tube causes a risk that the bending operation resulted in occurrence of uneven wrinkles of the outer tube or sandwiching of the outer tube between the plurality of bending pieces. In addition, the bending tube 2 is prone to a phenomenon in which the proximal end side is bent to a greater degree than the distal end side, sometimes making it difficult to perform a bending operation while maintaining the same radius of curvature.

In the bending tube 2 of the endoscope 1, the outer tube 30 includes a plurality of fixing regions 31 that are respectively adhesively fixed to the outer circumferential surfaces of the plurality of bending pieces 10. The fixing regions 31 are each ring-shaped, having, e.g., a width substantially equal to the width of each of the bending pieces 10 in the long axis direction.

The outer tube 30 is fixed in a state of being applied with a compressive force in the long axis direction between two adjacent fixing regions 31. In other words, the fixing regions 31 are formed in a state where the outer tube 30 is deformed due to application of a compressive force in the long axis direction. In other words, the outer tube 30a has a first length and a second length. The first length is between a pair of fixing regions 31 in the long axis direction. The second length is between a respective pair of circumferential regions in the long axis direction. The first length is greater than the second length. In other words, the outer tube 30a has a first diameter and a second diameter. The first diameter is a diameter of fixing regions 31. The second diameter is a diameter between a pair of fixing regions 31. The second diameter is greater than the first diameter.

As shown in FIGS. 4, 5, and 6A, the outer tube 30 is deflection-deformed and protrudes outward between the two fixing regions 31 even when the bending tube 2 is not bent (straight state). Therefore, the outer diameter of the outer tube 30 is greater than the outer diameter of the fixing regions 31 between the fixing regions 31.

The outer tube 30 that is an elastic member that is deflection-deformed generates a force that separates the two adjacent fixing regions 31 from each other in the long axis direction. Note that “elasticity” is the force that causes an object that has been deformed by an external force to return to the original state when the force is removed.

The restoring force of the outer tube 30 determines the respective relative positions of the plurality of bending pieces 10 in a state of being farthest from each other, causing the overall length of the bending tube 2 to automatically be a predetermined length.

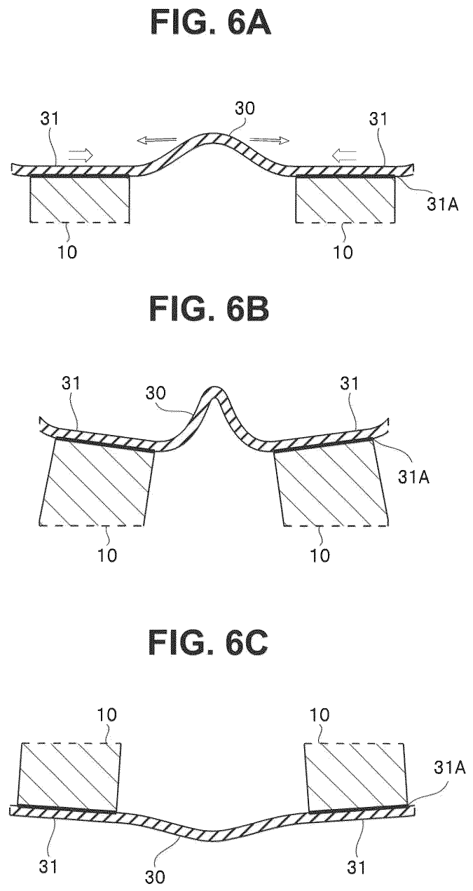

When the bending tube 2 is bent, the outer tube 30 between the two adjacent fixing regions 31 on the inside of the bending direction protrudes further outward from the state in FIG. 6A, as shown in FIG. 6B. This eliminates the risk of the outer tube 30 being sandwiched between the plurality of bending pieces 10. On the other hand, when the bending tube 2 is bent, the outer tube 30 on the outside of the bending direction protrudes less than in the state in FIG. 6A, as shown in FIG. 6C.

The bending tube 2 that is bent by the user's turn operation of the angle knob 6A automatically returns to a straight state due to the elastic restoring force of the outer tube 30 when the turning force is released. Since the turn operation in the opposite direction is not necessary, the endoscope 1 is easy to operate. In addition, the bending tube 2 with the plurality of bending pieces 10 fixed to the outer tube 30 is less likely to be in a twisted state.

As described above, the endoscope 1 of the embodiment is easy to manufacture and easy to operate.

Note that it is not industrially easy to determine from appearance alone whether between two adjacent fixing regions 31, the outer tube 30 is fixed in a protruding state due to the application of a compressive force in the long axis direction, or the outer tube 30 is fixed in a convex state in a stress-free state.

### Method for Manufacturing Bending Tube

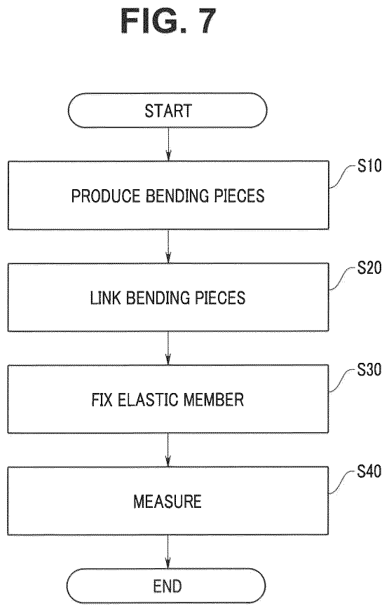

A method for manufacturing the bending tube 2 will be described in line with the flowchart shown in FIG. 7.

### <Step S10> Producing Bending Pieces

The plurality of bending pieces 10 are produced from a flat plate made of a metal such as a stainless steel or a flat plate made of resin by pressing or laser processing.

### <Step S20> Linking Bending Pieces

Using the plurality of hinges 20, the plurality of bending pieces 10 are linked with each other in a freely pivotable manner in the long axis direction, to thereby produce the bending tube main body 19.

### <Step S30> Fixing Elastic Member

The outer tube 30 is fixed to the outer circumferential surface of the bending tube main body 19 in a state where the outer tube 30 is deformed, applied with a compressive force in the long axis direction. The outer tube 30 deformed by the compressive force generates a force that separates the plurality of bending pieces 10 from each other in the long axis direction.

Specifically, e.g., while applying a tensile stress to the outer tube 30 in the long axis direction, each of the plurality of bending pieces 10 is adhesively fixed to each of the fixing regions 31 of the outer tube 30. Alternatively, each of the plurality of bending pieces 10 may be fixed to the outer tube 30 in a state where a tensile stress is applied to both ends of the bending tube main body 19 and the bending tube main body 19 is lengthened to the greatest extent. The outer tube 30 is fixed to the bending pieces 10 using an adhesive 31A, such as a thermosetting epoxy resin. If the bending pieces 10 are made of resin, the outer tube 30 and the bending pieces 10 may be welded together.

In order to efficiently apply a compressive force to the outer tube 30, the holes H12 and H13 into which the hinges 20 are to be inserted may have, instead of a circular the shape, a substantially ellipse shape or a track shape seen in track and field events, in which the dimension in the long axis direction of the bending tube main body 19 is longer than the dimension in the direction orthogonal to the long axis. A track shape is a rounded rectangle consisting of two parallel straight lines and two curved lines.

It is sufficient that the outer tube 30 is fixed to the bending piece 10 at the distalmost end (first end 2T1) and the bending piece 10 at the proximalmost end (second end 2T2).

The outer tube 30 can be further fixed to one of the bending pieces 10 in addition to the two bending pieces 10 mentioned above. In other words, the outer tube 30 includes a plurality of fixing regions 31 fixed respectively to two or more of the bending pieces 10 of the plurality of bending pieces 10.

The outer tube 30 may be fixed to at least one of the distal end portion 3 or the flexible tube 4 and to at least one bending piece 10.

Note that the outer tube 30 may be fixed to all of the bending pieces 10, but can be fixed to only one of the two adjacent bending pieces 10. In other words, the outer tube 30 can be fixed to only about half of the bending pieces 10 of the plurality of bending pieces 10 that configure the bending tube main body 19.

The endoscope 1 having the outer tube 30 fixed to only about half of all the bending pieces 10 requires less force for the bending operation and is easier to manufacture than an endoscope having the outer tube 30 fixed to all of the bending pieces 10.

The outer tube 30 can be made of a material with a large elastic limit that is deflection-deformed by compressive stress. The outer tube 30 is made of a resin (including rubber) having an elastic body, such as polyacetal, polycarbonate, nylon, polyethylene, polypropylene, fluororubber, urethane, raw rubber, EPDM (Ethylene Propylene Diene Monomer), EVA (Ethylen-Vinyl Acetate), or silicone resin.

The rubber exemplified as the material for the outer tube 30 is incompressible, but the elastic member to be fixed to the bending tube main body 19 may be compressible. For example, a compression spring may be fixed to the outer circumferential of the bending tube main body 19 as an elastic member. The elastic member may undergo compression deformation in which the elastic member is applied with a compressive stress to change in volume.

The elastic member applied with a compressive stress may simultaneously undergo both compression deformation with volume change and deflection deformation without volume change. For example, when applied with a compressive stress, a rubber with many air bubbles dispersed inside changes in volume due to compression deformation and, at the same time, is deflection-deformed.

### <Step S40> Measurement

An interval between the plurality of bending pieces 10 that are joined together is inspected as described below. The endoscope 1 is assembled using the bending piece bodies 19 having an interval that is within a predetermined range and that has passed the inspection.

The method for manufacturing the embodiment enables to easily manufacture the endoscope 1 that is easy to operate.

### Modification of First Embodiment

Endoscopes 1A-1F (bending tubes 2A-2F) of modifications of the first embodiment are similar to and have the same functions as the endoscope 1 (bending tube 2). Therefore, components having the same functions as those of the endoscope 1 are given the same reference numerals as those of the endoscope 1, and the description will be omitted.

### Modification 1 of First Embodiment

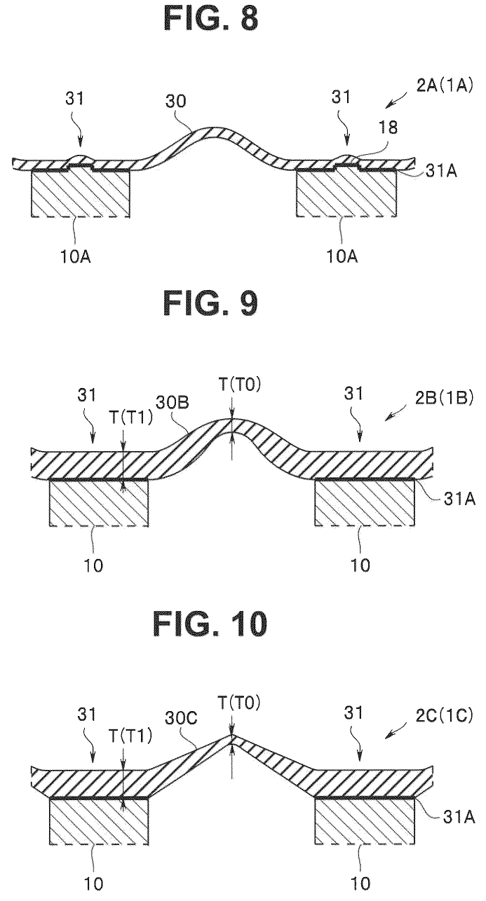

As shown in FIG. 8, in the bending tube 2A of the endoscope 1A of modification 1, bending pieces 10A to be fixed to the fixing regions 31 of the outer tube 30 each has a protrusion 18, which is a positioning member, that engages with each of the fixing regions 31. In other words, of the bending pieces 10A configuring the bending tube 2A, each of the bending pieces 10A that are respectively fixed to the fixing regions 31 of the outer tube 30 has a protrusion 18 that is a positioning member that engages with each of the fixing regions 31.

The protrusion 18 is, e.g., a ring-shaped wall along the outer circumference of the bending piece 10A. The outer tube 30 may have a recess into which the protrusion 18 of the bending piece 10A fits.

With the bending tube 2A, the outer tube 30 is more easily operated to fix to and more reliably fixable to the bending piece 10A while applying a compressive stress to the outer tube 30.

### Modifications 2 and 3 of First Embodiment

As shown in FIGS. 9 and 10, in bending tubes 2B and 2C of endoscopes 1B and 1C of modifications 2 and 3, outer tubes 30B and 30C have a thickness T0 between the two adjacent fixing regions 31 that is less than a thickness T1 of the two fixing regions 31. In other words, the region between the two fixing regions 31 is thin-walled.

The outer tube 30B of the bending tube 2B shown in FIG. 9 has a thickness T that is a minimum thickness T0 at the center of the two fixing regions 31, and the thickness T changes in a curved manner.

The outer tube 30C of the bending tube 2C shown in FIG. 10 has the thickness T that is the minimum thickness T0 at the center of the two fixing regions 31, and the thickness T changes linearly.

The bending tubes 2B and 2C are more likely to be deflection-deformed outwardly than the bending tube 2. For this reason, the bending tubes 2B and 2C are more likely than the bending tube 2 to generate a force that separates the two adjacent fixing regions 31 from each other in the long axis direction. In addition, the bending tubes 2B and 2C are less risky than the bending tube 2 to have the outer tubes 30B and 30C sandwiched between the plurality of bending pieces 10.

### Modification 4 of First Embodiment

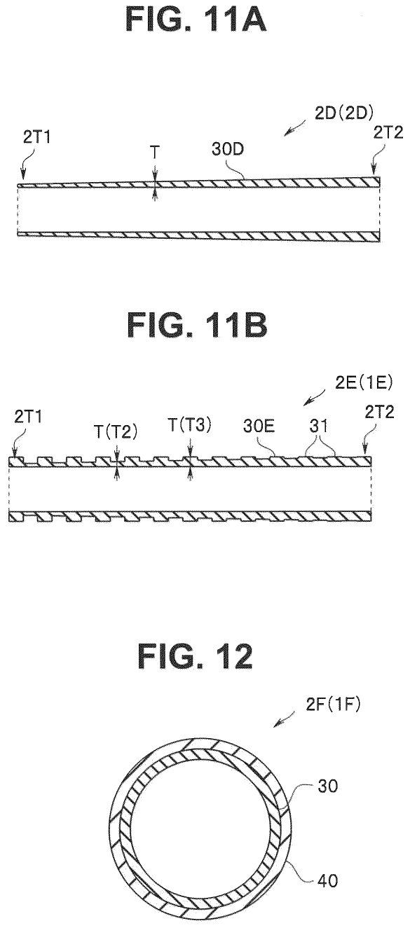

An outer tube 30D of a bending tube 2D of an endoscope 1D of modification 4 shown in FIG. 11A has a thickness T and therefore an elasticity (elastic modulus X thickness) that both decrease from the second end 2T2 to the first end 2T1 along the long axis direction. In other words, the elasticity is proportional to the elastic modulus (e.g., Young's modulus) of the material of the outer tube 30 and inversely proportional to the thickness of the outer tube 30.

Note that, as in the bending tube 2E of the endoscope 1E shown in FIG. 11B, a thickness T3 of the fixing regions 31 may be constant as long as the thickness T of the outer tube 30E is such that the thickness T2 between the fixing regions 31 decreases from the second end 2T2 to the first end 2T1. Between the respective fixing regions 31, the respective thicknesses T2 may not decrease from the second end 2T2 to the first end 2T1 and may be constant.

The bending tube 2, in which the operation wire 6B is fixed to the first end 2T1, may not be bent uniform the entirety since pulling the operation wire 6B can result in preferential bending of the region close to the second end 2T2.

In contrast, the bending tubes 2D and 2E of the endoscopes 1D and 1E have an elasticity that decreases from the second end 2T2 to the first end 2T1. For this reason, the bending tubes 2D and 2E tend to bend uniformly in the entirety since pulling the operation wire 6B can result in preferential bending of the region close to the first end 2T1.

Note that, as in the bending tubes 2B and 2C of the endoscopes 1B and 1C, the thickness T of the outer tubes 30D and 30E can decrease from the second end 2T2 to the first end 2T1, and the thickness T can be the minimum at the center of the two fixing regions.

### Modification 5 of First Embodiment

A bending tube 2F of an endoscope 1F of modification 5 shown in FIG. 12 includes a protective tube 40 that covers the outer tube 30, which is an elastic member, and the elasticity of the outer tube 30 is greater than the elasticity of the protective tube 40.

For example, if the outer tube 30 and the protective tube 40 are configured of a same material, the thickness of the outer tube 30 is greater than the thickness of the protective tube 40. The outer tube 30 and the protective tube 40 may be configured of different materials.

The endoscope 1F has the protective tube 40 and therefore has improved durability compared to the endoscope 1. The protective tube 40 does not cause significant reduction in the restoring force of the outer tube 30 that is deformed by a compressive stress.

### Second Embodiment

A bending tube 2G of an endoscope 1G of a second embodiment is similar to and has the same functions as the bending tube 2 of the endoscope 1. Therefore, components having the same functions as those of the endoscope 1 are given the same reference numerals as those of the endoscope 1, and the description will be omitted.

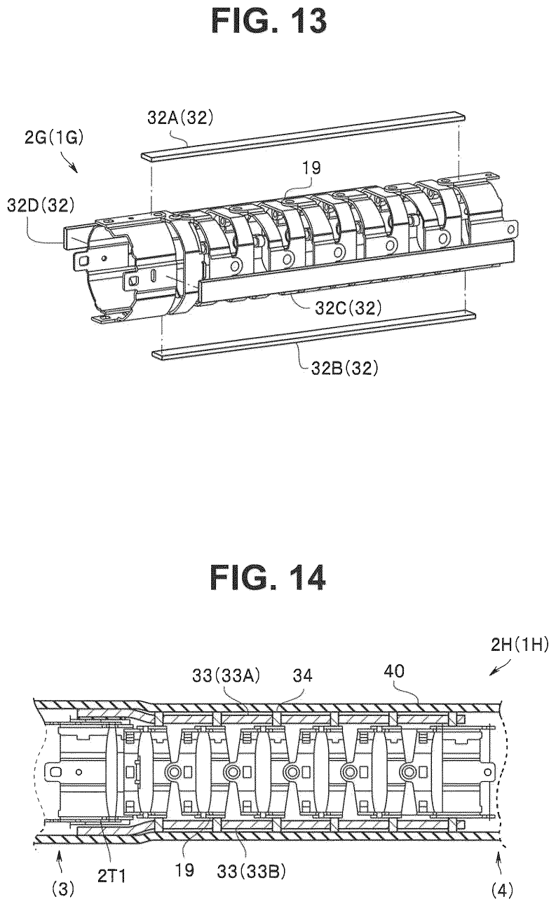

As shown in FIG. 13, the bending tube 2G includes rectangular rubber plates 32A, 32B, 32C, and 32D, which are elastic members, and the plurality of rubber plates 32 (32A-32D) are placed respectively in four directions (up/down/left/right) in which the bending tube 2G is to be bent. Each of the plurality of rubber plates 32 is adhered to the plurality of bending pieces 10.

Although not shown, the bending tube 2G including the rubber plate 32 includes the protective tube 40 as the bending tube 2F does. It goes without saying that the elasticity of the protective tube 40 is less than the elasticity of the rubber plate 32, which is an elastic member.

In the bending tube 2G, it is easier to fix the elastic member to the bending pieces 10 than in the bending tube 2, etc.

The thickness T of the rubber plate 32 can decrease from the second end 2T2 to the first end 2T1, and at the same time, the thickness T can be the minimum at the center of the two fixing regions 31.

### Third Embodiment

A bending tube 2H of an endoscope 1H of a third embodiment is similar to and has the same functions as the bending tube 2G of the endoscope 1G. Therefore, components having the same functions as those of the endoscope 1G are given the same reference numerals as those of the endoscope 1G, and the description will be omitted.

As shown in FIG. 14, in the bending tube 2H, rectangular leaf springs 33 (33A and 33B), which are elastic members, are disposed at rotationally symmetric positions on the outer circumferential surface of the bending tube main body 19.

Only a distal end of the leaf spring 33 is fixed to the bending piece 10 at the distalmost end (first end 2T1). As shown in FIG. 15, the bending piece 10 includes, on the outer circumferential surface, a bearing 34 that includes a hole H34 through which the leaf spring 33 is inserted. The leaf spring 33 is fixed to the bending tube main body 19 in a state to slide in the longitudinal direction when the bending tube 2 is bent.

When the bending tube 2H becomes a bent state, the leaf spring 33 slides in the longitudinal direction, and at the same time, moves in a direction orthogonal to the longitudinal direction (up and down directions in FIG. 15) in the space of the gap between an inner surface of the hole H34 of the bearing 34 and the leaf spring 33.

When the turn operation of the bending tube 2H is completed, the bending tube 2H automatically returns to a non-bent state (straight state) due to the elastic restoring force of the leaf spring 33. Therefore, when the endoscope 1H is operated, the bending tube 2H is bent as intended by the user. The endoscope 1H is able to prevent the protective cover from being caught between the bending pieces 10 when the bending tube 2H is bent.

Note that a compression spring may be disposed on a proximal end surface of the leaf spring 33. After being bent and deformed, the bending tube 2H returns to a straight state by the elastic restoring force due to the compression deformation of the compression spring in addition to the elastic restoring force due to the deflection deformation of the leaf spring 33.

### Fourth Embodiment

A bending tube 2I of an endoscope 1I of a fourth embodiment is similar to the bending tube 2H of the endoscope 1H and has the same function. Therefore, components having the same functions as those of the endoscope 1H are given the same reference numerals as those of the endoscope 1H, and the description will be omitted.

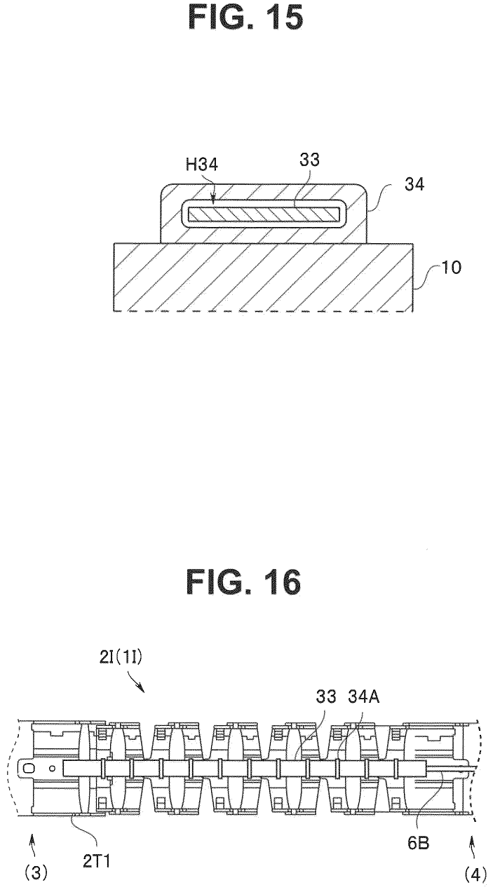

As shown in FIG. 16, in the bending tube 2I, the rectangular leaf springs 33, which are elastic members, are disposed at rotationally symmetric positions on an inner circumferential surface of the bending tube main body 19.

Only the distal end of the leaf spring 33 is fixed to the bending piece 10 at the distalmost end (first end 2T1). The bending piece 10 includes, on the inner circumferential surface, the bearing 34A that includes the hole H34 through which the leaf spring 33 is inserted. The bearing 34A has a configuration substantially the same as the bearing 34. The leaf spring 33 is fixed to the bending tube main body 19 in a state to slide in the longitudinal direction when the bending tube 2 is bent.

Each proximal end of the leaf springs 33 is connected to the operation wire 6B that is connected to the angle knob 6A. In other words, the leaf springs 33 each configure a part of the operation wire 6B that bends the bending tube 2I.

The foregoing described, as an example, a bending tube of an insertion portion of an endoscope. However, the bending tube of the embodiment may be a bending tube of a catheter, etc.

### Additional Notes

As already described, it is necessary to, after the bending tube 2 is manufactured, inspect whether the bending pieces 10 are placed at a predetermined interval. For example, an interval is measured between the plurality of wire guides 16 through each of which the operation wire 6B is inserted.

However, the endoscope 1 with the insertion portion 5 having a small diameter is not easy to inspect since the inner diameter of the bending tube 2 is small, e.g., within 10 mm.

There has been a demand for a method for manufacturing the endoscope 1 that is able to detect a plurality of structures placed in the bending tube 2 and accurately and easily calculate an interval between the plurality of structures.

Japanese Patent Publication No. H4-160303 discloses a measuring apparatus that inserts a laser distance meter into a pipe to measure an inner diameter of the pipe. However, as already described, it is not easy to insert a laser distance meter into a bending tube of an endoscope since the bending tube has a small inner diameter.

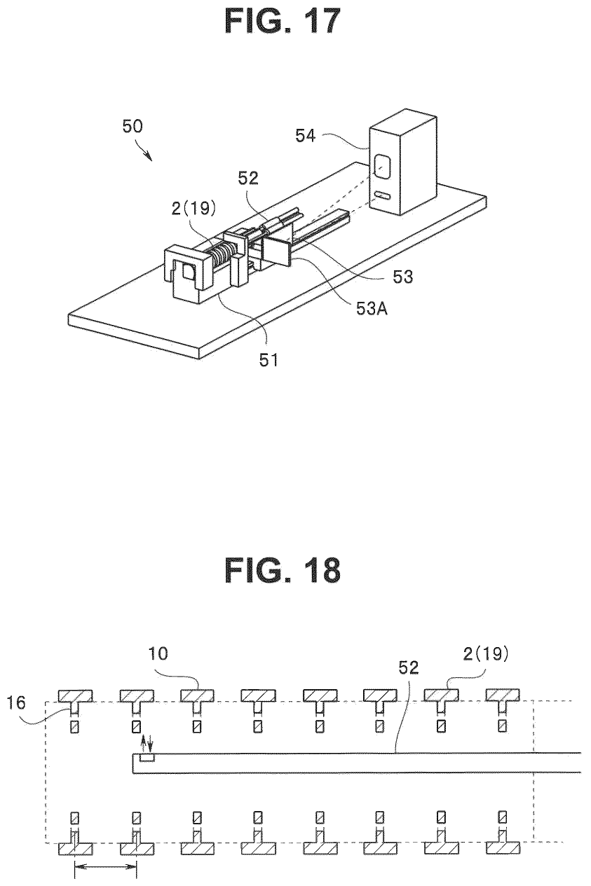

In a method for manufacturing the endoscope 1 of this reference example, a measuring apparatus 50 shown in FIG. 17 is used to measure the interval between internal structures, e.g., a plurality of wire guides 16, of the bending tube 2.

The measuring apparatus 50 includes: a fixing stage 51 on which the bending tube 2 (or the bending tube main body 19) is to be placed; a fiber sensor 52, which is an inspection tool, to be inserted into the bending tube 2; a moving stage 53 to which the fiber sensor 52 is fixed; and a distance meter 54.

The fiber sensor 52 includes an emission portion that emits light and a light receiving portion that receives light. The fiber sensor 52 includes a fiber having the emission portion and a fiber having the light receiving portion, both fibers being placed in a single sleeve. The moving stage 53 is configured such that the fiber sensor 52 is inserted into the bending tube 2 parallelly to the central axis of the bending tube 2. The distance meter 54 measures an amount of movement of the moving stage 53 by irradiating a reflector 53A of the moving stage 53 with laser light and detecting the return light.

As shown in FIGS. 18 and 19, the light emitted from the fiber sensor 52 is reflected by an inner surface of the bending tube 2, and the reflected light is received by the light receiving portion. The amount of light received by the light receiving portion is output as an electrical signal.

While light is emitted from the emission portion, the amount of light received by the light receiving portion is measured while the fiber sensor 52 is moved at a constant speed in a direction parallel to the central axis of the bending tube 2, and at the same time, the amount of movement is detected by the distance meter 54. The direction of movement may be a direction in which to insert or remove the fiber sensor 52 into or from the bending tube 2.

At positions where the wire guides 16 exist, the light emitted from the fiber sensor 52 is strongly reflected, and the strong reflected light is incident on the light receiving portion. Conversely, the reflected light is weak at positions where the wire guides 16 do not exist. Therefore, the amount of light (OUTPUT) received by the light receiving portion changes with the amount of movement.

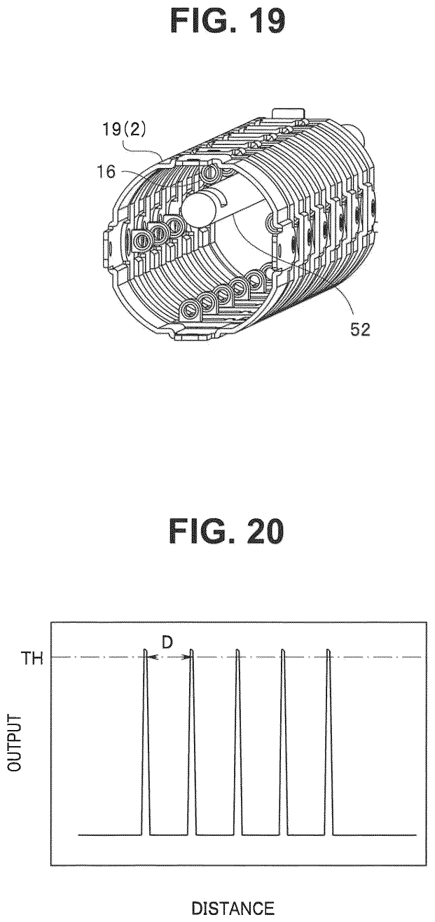

As shown in FIG. 20, a distance D between the wire guides 16 is measured based on a predetermined threshold value TH from the amount of movement (DISTANCE) measured by the distance meter 54 and the output (OUTPUT) of the fiber sensor 52.

The structures to be inspected may be the plurality of hinges 20, etc. To measure the amount of movement, a linear encoder may be used instead of the distance meter 54. The fiber sensor 52 may include an emission fiber and a light receiving fiber housed in separate sleeves. As the inspection tool, ultrasonic waves may be used instead of the fiber sensor 52 to measure the intensity of the reflected wave. The bending tube 2 may be fixed to the moving stage 53, and the fiber sensor 52 may be fixed to the fixing stage 51.

As described above, the method for manufacturing an endoscope includes the following configurations.
1. A method for manufacturing an endoscope, in which
   an inspection tool is moved inside a bending tube of an endoscope in a direction parallel to a long axis of the bending tube, while the inspection tool detects a plurality of structures placed in the bending tube, and based on a result of the detection, the interval between the plurality of structures is calculated.
2. The method for manufacturing an endoscope as described in 1 above, in which
   the plurality of structures are a plurality of wire guides that respectively hold a plurality of operation wires for bending the bending tube in a freely slidable manner.
3. The method for manufacturing an endoscope as described in 1 above, in which
   the plurality of structures are a plurality of hinges that connect a plurality of bending pieces included in the bending tube in a freely pivotable manner.
4. The method for manufacturing an endoscope as described in 1 above, in which
   the inspection tool is a fiber sensor, and positions of the plurality of structures in a long axis direction of the bending tube are detected based on emission and incidence of light.
5. The method for manufacturing an endoscope as described in 4 above, in which
   an interval between the plurality of structures is calculated based on a detection result that an intensity of the incidence is equal to or greater than a threshold value.
6. The method for manufacturing an endoscope as described in 1 above, in which
   the inspection tool is an ultrasonic probe, and positions of the plurality of structures in a long axis direction of the bending tube are detected based on transmission and reception of sound waves.
7. The method for manufacturing an endoscope as described in 6 above, in which
   the interval between the plurality of structures is calculated based on a detection result that the intensity of the reception is equal to or greater than a threshold value.
8. The method for manufacturing an endoscope as described in 1 above, in which
   an amount of movement of the inspection tool is measured by a distance meter, and
   an interval between the plurality of structures is calculated based on the amount of movement and the result of the detection.
9. The method for manufacturing an endoscope as described in 1 above, in which
   the inspection tool is inserted into or removed from the bending tube at a constant speed.

The present disclosure is not limited to the above-mentioned embodiments. etc., and various modifications, combinations, and applications are possible without departing from the gist of the disclosure.

What is claimed is:

1. An endoscope comprising:
a bending tube comprises:
a plurality of bending pieces;
a plurality of hinges linking two adjacent bending pieces of the plurality of bending pieces in a freely pivotable manner in a long axis direction; and
at least one elastic member including a plurality of fixing regions, fixed to a circumferential region of the plurality of bending pieces, respectively,
wherein a first length of the at least one elastic member between a pair of fixing regions in the long axis direction is greater than a second length between a respective pair of circumferential regions in the long axis direction.

2. The endoscope according to claim 1, wherein the plurality of fixing regions each fixed to an outer circumferential surface of the respective bending piece of the plurality of the bending pieces.

3. The endoscope according to claim 2, wherein the outer circumferential surface of the at least one elastic member is convex between adjacent pairs of bending pieces of the plurality of bending pieces.

4. The endoscope according to claim 1, wherein the plurality of fixing regions are fixed to corresponding circumferential regions of at least one adjacent pair of the plurality of bending pieces.

5. The endoscope according to claim 1, wherein the respective bending piece of the plurality of bending pieces includes a positioning member that engages with each of the plurality of fixing regions.

6. The endoscope according to claim 1, wherein the at least one elastic member is fixed to either one of the two adjacent bending pieces.

7. The endoscope according to claim 1, wherein the at least one elastic member has a thickness between two adjacent fixing regions of the plurality of fixing regions that is less than a thickness of the two fixing regions.

8. The endoscope according to claim 7, wherein the at least one elastic member has a thickness that is minimum at a center between the two adjacent fixing regions, and the thickness changes non-linearly.

9. The endoscope according to claim 7, wherein the at least one elastic member has a thickness that is minimum at a center between the two adjacent fixing regions, and the thickness changes linearly.

10. The endoscope according to claim 1, wherein the at least one elastic member has an elasticity that decreases from the second end to the first end along the long axis direction.

11. The endoscope according to claim 10, wherein the at least one elastic member has a thickness that decreases from the second end to the first end along the long axis direction.

12. The endoscope according to claim 1, further comprising:
a protective tube covering the at least one elastic member, wherein
an elasticity of the at least one elastic member is greater than an elasticity of the protective tube.

13. The endoscope according to claim 1, wherein the at least one elastic member is a tube made of a resin that covers outer circumferential surfaces of the plurality of bending pieces.

14. The endoscope according to claim 13, wherein the resin is rubber.

15. The endoscope according to claim 1, wherein
the at least one elastic member comprises a plurality of elastic members, the plurality of elastic members are placed respectively in a plurality of directions in which the bending tube is to be bent.

16. The endoscope according to claim 15, wherein each of the plurality of elastic members is a rectangular rubber plate.

17. The endoscope according to claim 15, wherein each of the plurality of elastic members is a leaf spring.

18. The endoscope according to claim 15, wherein
each of the plurality of elastic members is connected to a respective control wire of a plurality of control wires that bend the bending tube, and
a distal end of each of the plurality of elastic members is fixed to a bending piece of the plurality of bending pieces, the bending piece being placed at the second end.

19. A bending tube comprising:
a plurality of bending pieces;
a plurality of hinges that link two adjacent bending pieces of the plurality of bending pieces in a freely pivotable manner in a long axis direction; and
at least one elastic member including a plurality of fixing regions, fixed to a circumferential region of the plurality of bending pieces, respectively,
wherein a first length of the at least one elastic member between a pair of fixing regions in a long axis direction is greater than a second length between a respective pair of circumferential regions in the long axis direction.

20. A method for manufacturing a bending tube, the method comprising:
linking a plurality of bending pieces to each other in a freely pivotable manner in a long axis direction using a plurality of hinges, to produce a bending tube main body;
disposing at least one elastic member over the bending tube main body;
deforming the at least one clastic member by application of a compressive force in the long axis direction; and
while the at least one clastic member is deformed. fixing the at least one clastic member at two or more fixing regions to circumferential surfaces of respective bending pieces of the plurality of bending pieces.
